(19) United States
(12) Patent Application Publication
(10) Pub. No.: US 2012/0201883 A1
(43) Pub. Date: Aug. 9, 2012

(54) ANTIVIRAL COMPOSITONS

(75) Inventors: Elliot Hahn, Miami, FL (US); Robert I. Goldfarb, Miami, FL (US); Lawrence Solomon, Miami Beach, FL (US); Allan S. Kaplan, Boca Raton, FL (US)

(73) Assignee: ACCU-BREAK Technologies, Inc, Plantation, FL (US)

(21) Appl. No.: 12/963,803

(22) Filed: Dec. 9, 2010

Related U.S. Application Data
(63) Continuation-in-part of application No. 11/569,343, filed on Nov. 17, 2006, now Pat. No. 8,034,380.
Application No. 11/569,343 is a continuation-in-part of application No. 10/598,267, filed on Jun. 17, 2008, now abandoned.
Application No. 10/598,267 is a continuation-in-part of application No. 10/598,306, filed on Apr. 8, 2008, now abandoned.
(60) Provisional application No. 60/573,134, filed on May 21, 2004.
(60) Provisional application No. 60/573,134, filed on May 21, 2004.
(60) Provisional application No. 60/573,042, filed on May 21, 2004.

(51) Int. Cl. A61K 9/20; A61K 38/21; A61J 1/03; C07H 19/056; A61P 31/12; A61K 9/00; A61K 31/7056

(52) U.S. Cl. 424/465; 424/400; 424/85.4; 514/43; 536/28.7; 206/570

(57) ABSTRACT

A composition which includes a carboxamide, preferably ribavirin, for treating viral diseases in humans. A preferred embodiment of the subject invention comprises a very high dose (>600 mg) of ribavirin, and more preferably between about 800-1200 mg of ribavirin or more per dosage form.

### CROSS-REFERENCE TO RELATED APPLICATIONS

This is a continuation-in-part of U.S. patent application, Ser. No. 11/569,343, filed Nov. 17, 2006, which is a 371 filing from Int'l Application No. PCT/US05/18633 filed May 23, 2005 and which claims priority to U.S. Provisional Applications, Ser. Nos. 60/573,134 and 60/573,042, both filed May 21, 2004; this is also a continuation-in-part of U.S. patent application Ser. No. 10/598,267, filed Aug. 23, 2006, which is a 371 filing from Int'l Application No. PCT/US05/018638 filed May 23, 2005; this is also a continuation-in-part of U.S. patent application Ser. No. 10/598,306, filed Apr. 8, 2008, which is a 371 filing from Int'l Application No. PCT/US05/18639 filed May 23, 2005 and which claims priority to U.S. Provisional Applications, Ser. Nos. 60/573,134 and 60/573,042, both filed May 21, 2004, all of which are incorporated herein by reference in their entirety.

### BACKGROUND OF THE INVENTION

The compound, 1-B-D-ribofuranosyl-1,2,4-triazole-3-carboxamide, known as ribavirin, is described in U.S. Pat. No. 4,211,771. Ribavirin exhibits a wide spectrum of antiviral activity, both in vitro and in vivo. The antiviral properties of ribavirin, or its analogs, derivatives, isomers, polymorphs, or salts thereof, are utilized by administering an effective or therapeutic amount of the compound to human patients either by injection, orally, topically, ophthalmically, or via sprays or aerosols in the respiratory tract.

Ribavirin (marketed under the brand names Copegus, Rebetol, Ribasphere, Vilona and Virazole) is an anti-viral drug indicated for severe RSV infection (individually), hepatitis C infection (currently used in conjunction with peginterferon alfa-2b or peginterferon alfa-2a) and other viral infections. Ribavirin is a prodrug which, when metabolized, resembles purine RNA nucleotides. In this form it interferes with RNA metabolism required for viral replication. The specific mode of action and how ribavirin affects viral replication is unknown; multiple mechanisms may be responsible for its actions.

Physically, ribavirin is similar to the sugar D-ribose from which it is derived. It is freely soluble in water, and is re-crystallized as fine silvery needles from boiling methanol. The three free sugar hydroxyls make the pure drug hydrophilic enough that it is only sparingly soluble in anhydrous ethanol.

Ribavirin can be prepared from natural D-ribose by blocking the 2′, 3′ and 5′-OH groups with benzyl groups, then derivatizing the 1′-OH with an acetyl group which acts as a suitable leaving group upon nucleophilic attack. The ribose 1′ carbon attack is accomplished with a 1,2,4 triazole-3-carboxymethyl ester, which directly attaches the 1′ nitrogen of the triazole to the 1′ carbon of the ribose, in the proper 1-β-D isomeric position. The bulky benzyl groups hinder attack at the other sugar carbons. Following purification of this intermediate, treatment with ammonia in methanolic conditions then simultaneously deblocks the ribose hydroxyls, and converts the triazole carboxymethyl ester to the carboxamide. Following this step, ribavirin may be recovered in good quantity by cooling and crystallization.

Ribavirin can be viewed as a ribosyl purine analogue with an incomplete purine 6-membered ring. Such 5′ imidazole riboside derivatives show antiviral activity with 5′ hydrogen or halide, but are generally less active than ribavirin. Two natural products are known with this imidazole riboside structure: pyrazomycin/pyrazofurin, an antibiotic with antiviral properties, and the natural purine synthetic precursor 5-aminoimidazole-4-carboxamide-1-β-ribofuranoside (AICAR).

Taribavirin (previously known as viramidine and ribamidine) is a known 3-carboxamidine derivative of ribavirin. This drug shows a similar spectrum of antiviral activity to ribavirin, and is now known to be a pro-drug for ribavirin. Viramidine, however, has useful properties of less erythrocyte-trapping and better liver-targeting than ribavirin.

Ribavirin tablets or caplets can be made using a dry compaction process, as described in U.S. Pat. Nos. 6,051,252, 5,916,594 and 5,914,128. Each of U.S. Pat. Nos. 6,051,252, 5,916,594 and 5,914,128 describes a method of producing dosages of ribavirin using high pressures which could generate high temperatures, thereby reducing the efficacy or shelf-life of the active ingredient.

The manufacture of particles or pellets comprising ribavirin, using a wet granulation process for use in capsules, is described in US Pat. Nos. 7,538,094 and 6,720,000. Oral dosage forms, which include large doses (400-600 mg) of ribavirin in a single or exactly one dosage form, administered as whole tablets once or twice daily, are also described for increasing patient compliance (US Publ'n Nos. 2010/0203127 and 2009/0012015).

Despite these disclosures, there remains a need for oral dosage forms comprising greater than 600 mg of ribavirin. There is yet a further need for providing ribavirin in divisible tablet dosage forms. Conventional dosage forms, which can be divisible, are known and can be scored to facilitate dividing the dose by breaking through the dosage form at the position of the score. In addition, compressed tablets having an inactive layer or segment formed proximate to, or between two or more active layers or segments, are described in U.S. Pat. Nos. 7,329,418, 7,318,935, and US Publ'n No. 2008/0233190, which are incorporated herein by reference in their entirety.

Dosage forms comprising greater than 600 mg of ribavirin or comprising ribavirin in a segmented divisible tablet are not previously described. Oral dosage forms comprising greater than 600 mg, and segmented divisible tablet dosage forms comprising ribavirin, can provide greater dosing flexibility, save time and expense by decreasing the number of doctor's office visits or prescriptions required, and further advance patient compliance.

### SUMMARY OF THE INVENTION

The present invention relates to a composition which includes a carboxamide, preferably ribavirin, for managing or treating viral diseases in humans. A preferred embodiment of the subject invention comprises a very high dose (>600 mg) of ribavirin, and more preferably between about 800-1200 mg of ribavirin or more per dosage form.

In another embodiment of the invention, an effective amount of a carboxamide, e.g., ribavirin, is provided in a divisible dosage form, preferably a compressed tablet, and more preferably in a segmented (layered) tablet having an inactive breaking region. The dosage form can further comprise one or more additional active pharmaceutical ingredients (API) which is not ribavirin. Preferably, the one or more additional APIs have antiviral activity. A preferred divisible dosage form is a compressed tablet configured in a layered or segmented fashion, wherein the tablet comprises at least one active layer or segment and at least one inactive layer or segment, wherein the inactive segment forms a breaking segment or region to divide the dosage form therethrough, without breaking through or otherwise negatively affecting the integrity of an active segment.

The divisible compressed tablet can, in one embodiment, have a height greater than its width, the height being the vertical measurement of the tablet and the width being the horizontal measurement of the tablet as it is oriented in a tablet die during compression, but before ejection from the tablet die. A preferred tablet having its height greater than its width (i.e., a taller-than-wide tablet) can comprise two active layers or segments (containing an effective amount of an API, and an inactive layer or segment (containing less than an effective amount of API) formed or disposed between the two active layers/segments. The active layers or segments can each preferably comprise from about 400 mg to about 600 mg ribavirin, resulting in the whole tablet containing from about 800 to about 1200 mg of ribavirin. Preferred segmented or layered dosage forms according to the subject invention comprise 800 mg, divisible into two tablet halves comprising 400 mg each; comprise 1000 mg, divisible into tablet halves comprising 500 mg each; or comprise 1200 mg, divisible into tablet halves comprising 600 mg each.

A segmented tablet of the subject invention can also contain one or more additional APIs, either combined with a carboximide, e.g., ribavirin, in one or more of the active layers or segments, or can comprise a carboximide such as ribavirin in one active layer or segment, and one or more additional APIs, preferably at least one antiviral API, in the other active layer or segment.

The dosage forms of the subject invention can be useful in a method of managing or treating a viral condition or disease in an animal, e.g., a human, comprising the steps of:
a) providing a single or exactly one whole divisible dosage form according to the subject invention,
b) breaking the whole dosage form to provide two halves, and
c) administering to a patient, or directing the patient to administer, one of the two halves of the divided dosage form.

The method can further comprise the step of administering the remaining half at a later time, preferably up to about 18 hours after administration of the first half, more preferably from about 6 to about 12 hours after administration of the first half, and most preferably between about 8 and about 12 hours after administration of the first half.

A further embodiment of the invention comprises providing a dosage form of the subject invention in a kit for management or treatment of a viral condition or disease. A kit according to the invention comprises a first dosage form, e.g., a compressed taller-than-wide tablet, as described and defined herein, comprising a carboxamide compound such as ribavirin, which is effective to manage or treat a viral condition or disease in a patient, and at least one additional dosage form comprising one or more APIs, which are preferably different than the carboximide compound of the first dosage form. The kit can further comprise instructions for use or administration of the first and the additional dosage form or forms. The additional dosage form(s) included in the kit can be a segmented tablet or a conventional tablet or capsule comprising a known or novel antiviral compound. For example, the kit can comprise a first tablet comprising greater than 600 mg ribavirin and a second tablet comprising less than 600 mg ribavirin or a tablet comprising greater than 600 mg having a strength different than the first tablet. Alternatively, the one or more additional dosage forms included in the kit can comprise one or more APIs which are different than the active contained in the first tablet, and can have antiviral activity or can have a property useful for treatment of a different (non-viral) condition or disease, such as an antibiotic active against bacterial infection.

### DETAILED DESCRIPTION

The invention concerns a novel dosage form comprising a carboxamide compound, e.g., ribavirin, or its analogs, derivatives, isomers, polymorphs, or salts, used as an active pharmaceutical ingredient (API) in the dosage form.

Ribavirin is preferably provided in the dosage form at a strength which is higher than provided in previously known ribavirin dosage forms. The highest known strength of a marketed ribavirin product is a tablet containing 600 mg of ribavirin. Embodiments of dosage forms of the subject invention can contain ribavirin at strengths of greater than 600 mg, preferably at least about 800 mg and more preferably between about 800 mg to about 1200 mg. The subject invention can further include a high-dose ribavirin tablet provided with a score, such as a bisecting score, or a novel scoring pattern such as a trisecting, quadrisecting or pentasecting score to enable unique dosing regimens for ribavirin.

Tablets of the subject invention are formulated to include, in addition to ribavirin as the API, pharmaceutically acceptable excipients that are well known in the art. These excipients are formulated with the API into dosage forms, e.g., tablets, using known pharmaceutical formulation and manufacturing procedures as described in, for example, Remington's Pharmaceutical Sciences 20th Ed., Mack Publishing Co., Easton, Pa. (2000), Chapter 45, which is incorporated by reference.

In a further embodiment of the subject invention, the dosage form can include ribavirin in combination with one or more APIs. In one preferred embodiment, a second API is another antiviral drug other than ribavirin. Alternatively, the additional APIs can be a drug or drugs having therapeutic use other than antiviral activity.

In addition, dosage forms of the subject invention can be formulated as layered tablets using accurately breakable tablet technologies, as disclosed in Int'l Applications WO 2005/112,870; WO 2005/112,897; WO 2005/112,898; WO 2005/112,900; WO 2006/038,916; and US 2006/0003000, which are also incorporated herein by reference in their entirety.

Novel methods of using a carboximide compound, e.g., ribavirin, including a method of treating a patient for a viral condition or disease using a tablet containing greater than 600 mg of ribavirin, and a method of treating a patient using a portion of a divisible layered or segmented tablet, are part of the subject invention.

In use, novel tablets of the subject invention can be broken to provide a portion of the whole tablet to achieve a standard daily dose of ribavirin from a single tablet. This capability provides an advantage to a physician and a patient of being able to provide, for example, an initial dose of ribavirin by administering only a half tablet. Such treatments previously required use of a whole tablet for each dose.

The method of the subject invention can advantageously reduce the number of doctor visits and prescriptions and can provide greater dose flexibility for the physician. In addition, a higher initial dose (one whole tablet plus a portion of a broken or divided tablet) can also be provided to effect a desired response in accordance with a physician instruction. Moreover, dosing flexibility or adjustability during a treatment plan can be achieved by providing the subject tablets that are divisible by a patient or caregiver in situations where too high a dose may cause undesired side effects, or when dosage needs to be increased to effect a desired response, such as more rapid onset of effect or to facilitate getting a patient to a goal endpoint. Intermediate dosage strengths can also be an advantage of the subject divisible tablets.

Tablets of the invention are preferably those compressed in a tablet press. For commercial use, a high-speed three (3)- or five (5)-layer press produced by Korsch AG may be utilized. The tablets of the invention are primarily intended for oral administration but they may also be used for other applications. Tablets of the invention are not formed using a cement, glue, adhesive, or the like, and can be coated or uncoated. The tablets of the invention can comprise at least two compositionally different segments.

A segment represents the entirety of a contiguous, substantially homogeneous part of a tablet or tablette (see below) of the invention. If two or more consecutive granulations entering the die are substantially identical, then when compressed, they will form one segment. Such a segment is a sub-type of segment that may be referred to specifically as a compound segment. If, however, two substantially non-identical granulations (such as those containing different active drugs, the same active drugs in different ratios, different excipients or different ratios of similar excipients, or different salts of the same active drug) were compressed onto each other, they would comprise two segments. Granulations comprising the same active drug in the same concentration relative to excipients but with dissimilar excipients would comprise two segments if one granulation were compressed onto another.

A layer is produced by introducing an amount of an individual granulation into a tablet die to fill at least a part of the die. A layer is considered to be present whether it is the form of all un-tamped, tamped or fully compressed granulation.

In many of the most preferred tablets of the invention, a layer (and the granulation from which it is derived) will not need to be placed on top of or below (e.g., adjoining, or contiguous with) a substantially identical layer (or granulation). In such a case, one layer will give rise to the sub-type of segment that is a simple segment. The use of the term “segment” allows a segment to be simple (single layer forming a single segment or compound (a single segment formed by more than one layer of the same composition.) Because the tablets of the invention have been adapted to be broken if and when desired, it has proven useful to develop a term for the major fragments obtained from said breaking. The inventors use the term “tablette” in this regard. An example of tablette formation is as follows: when a standard single-scored, mono-layer, homogeneous pharmaceutical tablet is broken, said breaking produces two major fragments, each of which is called a tablette. However, this breaking of a conventional tablet, generally, produces some chips and crumbling in the active-containing portion of the tablettes, thus creating some loss of active ingredient in the administered dose. In the segmented, layered tablets of the invention having an inactive breaking segment, successfully breaking said tablet through said inactive segment will result in two tablettes, without any crumbling or chipping occurring in or from the active segment.

The terms “active agent,” “drug,” “active drug,” “active pharmaceutical agent,” and “pharmacologically active agent” are interchangeable and include, without limitation, prescription and non-prescription pharmaceutical compounds, as well as pharmacologically effective doses of vitamins cofactors, and the like. Substances such as foodstuffs, vitamins in “recommended daily allow” quantities, and the like are not considered to be “drugs” herein.

The term “undetectable amount” means that the presence of an active compound cannot be identified when using conventional analytical techniques such as high performance liquid chromatography (HPLC), nuclear magnetic resonance imaging (NMRI), and the like. The term “pharmacologically ineffective amount” means an amount of a drug or drugs that has no measurable pharmacological effect. Due to the conditions under which high speed automated tabletting equipment are operated, mixing of different granulations may occur during tablet formation which may cause material such as drug substance present in one granulation to appear in a layer or segment where it was not intended to be placed.

The term “inactive segment” refers to a segment that either contains an undetectable amount of any drug or contains a diminished concentration of any pharmacologically effective drug or drugs contained in another segment or segments.

The compositions forming the active and inactive segments can include pharmaceutically acceptable excipients which are well known in the art. These excipients can be used in conventional manufacturing and processing methods to form immediate-release, controlled-release (including sustained release, delayed release, or fast dissolving formulations or the like). Excipients can be characterized according to their function during the formulation as, for instance, binders, disintegrants, fillers (or diluents), glidants, lubricants and eventually flavors, sweeteners and dyes.

Lubricants are intended to improve the ejection of the compressed tablet from the die of the tablet-making equipment. Glidants are added to improve the powder flow. They are typically used to help the mixture of all the components to fill evenly and regularly the die before the compression. Fillers are inert ingredients sometimes used as bulking agents in order to decrease the concentration of the active ingredient in the final formulation. The binders in many cases also provide the function of a filler. Disintegrants may be added to formulations in order to help the tablets disintegrate when they are placed in a liquid environment and so release the API. The disintegration properties are, mostly, based upon the ability of the disintegrant to swell in the presence of a fluid, such as water or gastric juice. This swelling disrupts the continuity of the tablet structure and thus, allows the different components to enter into solution or into suspension. Commonly used disintegrants include native starches, modified starches, modified cellulose, microcrystalline cellulose or alginates. Binders are used to hold together the structure of the dosage forms. They have the property to bind together all the other ingredients after sufficient compression forces have been applied and they provide the integrity of the tablets. Starches are known to act in some cases as binders and in some other cases as disintegrants according to the fact that they are native, chemically modified or physically modified.

As an example of a method of manufacture of a preferred tablet of the invention, first, a granulation containing a pharmacologically effective dose of a drug enters the die and is tamped. Second, a granulation lacking a drug (an “inactive granulation”) enters the die and is tamped. Optionally, another inactive granulation lacking active drug also enters the die and is tamped. The inactive granulation(s) creates a part of the tablet that can be identified and broken through so that a part of the drug containing a significant concentration of drug is not broken through. Last, a second granulation containing a pharmacologically effective quantity of a drug enters the die, is optionally tamped, and then final compression to form a compressed tablet occurs. While one or all segments may individually have a width greater than height, the tablet as a whole has a height that exceeds its width.

Subsequent to tablet formation, optionally a score may be placed in the side of said tablet, preferably transversely. Alternatively, after tablet formation, a printed line or other forms of indicia such as dotted lines, symbols or perforations may be placed on or in the surface of the tablet, all of which serve the purpose of allowing identification of said tablet's desired breaking region from the standpoint of effecting accurate separation of the parts of a tablet containing isolated doses of drug. Other means of aiding identification of a region of potentially desired tablet breaking may be utilized such as the use of contrasting colors in different segments.

Suitable dimensions for tablets according to the invention are: height: 6 to 24 mm; preferably 10 to 18 mm and more preferably from 10 to 14 mm; width (at the widest dimension of the horizontal axis): 2 to 16 mm: preferably 3 to 10 mm and more preferably 4 to 8 mm. Without limitation, the dimensions of the tablet may be optimal if the ratio of the height to the width is between about 1.5:1 to about 3:1.

Tablets of the invention are most preferably formed in a high-speed tablet press. In a typical manufacturing procedure, two or more different granulations are separately fed into a die, utilizing different filling stations. Wet granulations are often preferred to limit transfer of material from one granulation to another. Direct compression of powder is also a preferred manufacturing technique.

Full entry of a granulation at a filling station produces a layer. Tablets of the invention have a layered structure. It may be relevant to add two consecutive substantially identical inactive granulations to form two identical, contiguous layers. After compression, the part of the tablet formed by said two identical granulations is referred to as a segment. The type of segment so formed is called a compound segment. There also may, in the case in which a large quantity of an API is desired to be in one segment, since the two have substantially identical granulations containing said API which enter at two consecutive filling stations, thus producing, after final compression a tablet containing, a compound segment that contains the API. The more common case is that in which a first granulation enters the die not on top of a substantially identical granulation (layer), forms a layer, and a substantially non-identical (i.e., different) granulation next enters said die, so that said first granulation forms a layer that is not contiguous with a substantially identical layer. Said first granulation forms a layer that is considered to be a simple segment in the compressed tablet.

The benefits of the invention include the utilization preferably of inactive granulations and less preferably, segments with diminished concentration of a drug relative to another segment. Optimally, the tablet is provided with a means of identifying an optimal breaking region and of identifying one tablette from another after tablet breaking, an important benefit if the tablettes contain different types or quantities of a drug or drugs.

Because of the novelty of the tablets, it is necessary to describe the top, bottom, sides, etc. of the tablet. It has been found best to describe the tablet with regard to such terms based on said tablet's formation and location in the die in which said tablet is formed.

As used herein, such terms as “horizontal” (“transverse”) and “vertical” when used in relation to a tablet, are based on the spatial orientation of the tablet as, and after, it is produced in a die, but before removal or ejection from the die. Current methods of manufacture produce tablets with one granulation entering the die on top of another, so that tablets of the invention produced in such a manner comprise one or more top (outer) segments, one or more bottom (outer) segments, and optionally one or more middle (inner) segments. A segment that is not a top or bottom (collectively, “outer”) segment is considered to be an inner segment, even though of course an inner segment has an external aspect. The number of inner segments is not limited.

The bottom segment of a tablet contains the first granulation to enter the die. The top segment of a tablet contains the last granulation to enter the die. A “side” of the tablet refers to that external part of said tablet in contact with the internal vertical face or aspect of the tablet die in which said tablet is produced. Typically, sides of the tablets of the invention are vertically oriented, in contrast to the tops and bottoms of the invention. In the case of cupping and beveling of the top of the tablet, which may from time to time be extensive, the tablet's side is considered to also include the external part of the tablet that was in contact with the internal vertical face or aspect of the tablet die before a top punch formed said cupping, beveling, or the like.

If separate granulations were to be sequentially placed in a die horizontally (side-to-side) and not vertically as is currently the practice, then the tablets so produced would be within the scope of the present invention as the same product would be produced. When the tablet of FIG. 1, for example, is laid on a flat table, it will tend to lie lengthwise at right angles to the manner in which it is formed in the die (i.e., its longest axis would lie horizontally in relation to the tabletop), so that if the three segments were all different colors, then the segments would appear to be arranged not vertically (one on top of the other), but rather horizontally (side-to-side). For consistency of terminology, such segments nonetheless are considered herein to be disposed vertically on top of each other, because of the manner in which they were created.

One advantage of the invention is that it optimizes optional tablet breaking. When force is applied to break a tablet, breaking of the tablet tends to more easily produce predictable quantities of the API or APIs in tablettes than “wider than tall” tablets with segments containing the same quantities of drugs. The tablet may be broken according to the invention either by applying force such as a cutting edge directly to the region to be broken through, or to outer segments, potentially in either case breaking through an inner segment.

The practice of the present invention employs, unless otherwise indicated, conventional techniques of drug manufacture and administration, which are well known in the art.

### EXAMPLE OF MANUFACTURING PREFERRED EMBODIMENTS

A “taller than wide” tablet is made which has three segments, each with an active top or upper segment (containing an API) and an active lower or bottom segment (containing an API) separated by a substantially inactive middle segment. A Korsch multi-station, multi-layer rotary tablet press can be used. All formulations can be achieved by those skilled in the pharmaceutical manufacturing arts. The blending both of the API (e.g., ribavirin) formulation can be performed in a shaped blender. The middle segment can comprise inactive pharmaceutically acceptable excipients, e.g., 194 mg of Nu-Tab™, microcrystalline cellulose (MCC), or sugars such as lactose, or the like, and requires no blending.

The tablets can be compressed using 0.131 inch by 0.3222 inch oval, concave tablet punches to a hardness of 35 kiloponds. The bottom segment is introduced first into the die. The tablet weight is 310 mg. Tablets so made are 8 mm tall; the inactive middle segment varies from 5-6 mm in height and a width of 4 mm. Weights in mg of the granulation comprising each segment are as follows:

Bottom Segment: Dibasic calcium phosphate anhydrous 51.13 mg; ribavirin 400-600 mg; Sodium starch glycolate (Explotab™) 2.48 mg; Magnesium stearate 0.93 mg; and FD&C Blue #1 Aluminum Lake 0.31 mg.

Middle Segment: Nu-Tab™ (compressible sugar 30/35 N.F.), 194.00 mg, or alternatively, Dibasic calcium phosphate anhydrous, 158.59 mg; Magnesium stearate 2.79 mg; and PVP K-30, 2.62 mg.

Top Segment: Dibasic calcium phosphate anhydrous 51.13 mg; ribavirin 400-600 mg; Sodium starch glycolate (Explotab™) 2.48 mg; Magnesium stearate 0.93 mg; and FD&C Blue #1 Aluminum Lake 0.31 mg.

Manufacturing Instructions
1. Weigh each ingredient.
2. Screen each ingredient.
3. Triturate the color with the major diluent in geometric proportions using a suitable mixer.
4. Add the remaining ingredients except the lubricants to the color mixer from Step #3 and mix for desired time.
5. Add the lubricant to the blend from Step #4 and mix for desired time.
6. Add the blend to a suitable press fitted with the desired tooling and compress into tablets.

Tabletting Instructions
1. Place the powder for active layer in hopper #1.
2. Place the powder for placebo layer in hopper #2.
3. Place the powder for active layer in hopper #3.
4. Compress layer #1 tablets to desired weight (tablets for layer #1 should form a soft compact).
5. Compress layer #1 & layer #2 tablets to desired combined weight of layer #1 and layer #2 weight (tablets should form a soft compact).
6. Compress the multi-layer tablet to the desired total tablet weight (layer #1 weight+layer #2 weight+layer #3 weight). Tablet should be at desired hardness.

A similar tablet of the invention can be separately produced using different actives in each of the top and bottom segments, respectively.

The invention may be embodied in other specific forms without departing from the spirit or essential characteristics thereof. The foregoing embodiments are therefore to be considered in all respects illustrative rather than limiting of the invention described herein. Scope of the invention is thus indicated by the appended claims rather than by the foregoing description, and all changes that come within the meaning and range of equivalency of the claims are therefore intended to be embraced herein.

1. A dosage form comprising a carboxamide compound, or a derivative, isomer, polymorph, or salt thereof, in an amount greater than 600 mg.

2. The dosage form of claim 1, wherein the carboxamide is ribavirin, or a derivative, isomer, polymorph, or salt thereof.

3. The dosage form of claim 2, wherein the ribavirin is present in an amount between about 800 mg and about 1200 mg.

4. The dosage form of claim 3 wherein the ribavirin is present in an amount of about 800 mg.

5. The dosage form of claim 3 wherein the ribavirin is present in an amount of about 1000 mg.

6. The dosage form of claim 3 wherein the ribavirin is present in an amount of about 1200 mg.

7. The dosage form of claim 3, wherein the dosage form is a divisible tablet or caplet.

8. The dosage form of claim 2 wherein the dosage form comprises one or more additional API(s) which is not ribavirin.

9. The dosage form of claim 5 wherein a second active pharmaceutical ingredient has antiviral activity.

10. A divisible dosage form comprising a carboximide compound, or a derivative, isomer, polymorph, or salt thereof, said dosage form having at least one active layer or segment and at least one inactive layer or segment, said inactive layer or segment forming a breaking segment to divide the dosage form therethrough, without breaking through an active segment.

11. The divisible dosage form of claim 10, wherein the dosage form is a compressed tablet.

12. The divisible dosage form of claim 11 wherein the compressed tablet has a height and width, wherein the height of said tablet is greater than the width of said tablet, said height being the vertical measurement of the tablet and said width being the horizontal measurement of the tablet as the tablet is oriented in a tablet die during compression but before ejection from the tablet die.

13. The divisible dosage form of claim 12 wherein the tablet comprises two active layers or segments, at least one of the active layers or segments comprising ribavirin, and an inactive layer or segment formed between the two active layers or segments.

14. The divisible dosage form of claim 13 wherein the tablet comprises two active layers or segments, each active layer or segment comprising between about 400 mg to about 600 mg ribavirin, and an inactive layer or segment formed between the two active layers or segments.

15. The divisible dosage form of claim 13 wherein at least one active layer comprises ribavirin, and at least one active layer comprises one or more API(s) which is/are not ribavirin.

16. The divisible dosage form of clam 15 wherein the API(s) which is/are not ribavirin has/have antiviral activity.

17. A method of managing or treating a viral disease or condition in a human, said method comprising the steps of:
a. Providing a single or exactly one whole divisible dosage form of claim 1
b. Breaking the whole dosage form to provide two halves of said dosage form, and
c. Administering or directing a patient to administer only one of the two halves of the dosage form to the patient.

18. The method of claim 17 wherein the whole dosage form comprises between about 800 mg to about 1200 mg ribavirin and each tablet half comprises from about 400 mg to about 600 mg of ribavirin.

19. The method of claim 17 wherein the whole dosage form is a compressed tablet having a height and width, wherein the height of said tablet is greater than the width of said tablet, said height being the vertical measurement of the tablet and said width being the horizontal measurement of the tablet as the tablet is oriented in a tablet die during compression but before ejection from the tablet die.

20. The method of claim 17 wherein the whole dosage form is a compressed tablet comprising two active layers or segments, at least one of the active layers or segments comprising ribavirin, and an inactive layer or segment formed between the two active layers or segments.

21. The method of claim 17 wherein the method further comprises the step:
administering a remaining half of the divided dosage form at a later time.

22. The method of claim 21, wherein the later time is between about 6 hours and about 12 hours after the first administration of a half of the divided dosage form.

23. A kit for management or treatment of a viral condition, said kit comprising:
a compressed tablet having a height and width, wherein the height of said tablet is greater than the width of said tablet, said height being the vertical measurement of the tablet and said width being the horizontal measurement of the tablet as the tablet is oriented in a tablet die during compression but before ejection from the tablet die, said tablet comprising two active layers or segments, at least one active layer or segment comprising ribavirin, and an inactive layer or segment formed between the two active layers or segments;
a second dosage form comprising one or more API(s); and
instructions for use or administration of the solid dosage form and the second dosage form;
wherein, the solid dosage form and the second dosage form are packaged in separate compartments or areas of a single packaging unit.

24. The kit of claim 23 wherein the second dosage form comprises one or more antiviral compounds.

25. The kit of claim 23 wherein the packaging unit is a blister pack.
